(19) United States
(12) Patent Application Publication
(10) Pub. No.: US 2018/0169095 A1
(43) Pub. Date: Jun. 21, 2018

(54) THE USE OF SGC STIMULATORS, SGC ACTIVATORS, ALONE AND COMBINATIONS WITH PDE5 INHIBITORS FOR THE TREATMENT OF DIGITAL ULCERS (DU) CONCOMITANT TO SYSTEMIC SCLEROSIS (SSC)

(71) Applicant: Bayer Pharma Aktiengesellschaft, Berlin (DE)

(72) Inventors: Claudia HIRTH-DIETRICH, Wuppertal (DE); Peter SANDNER, Wuppertal (DE); Johannes-Peter STASCH, Grottaferrata (RM) (IT); Michael HAHN, Langenfeld (DE); Markus FOLLMANN, Köln (DE); Alexandros VAKALOPOULOS, Hilden (DE)

(73) Assignee: Bayer Pharma Aktiengesellschaft, Berlin (DE)

(21) Appl. No.: 15/571,105

(22) PCT Filed: May 2, 2016
(86) PCT No.: PCT/EP2016/059734

§ 371 Date: Nov. 1, 2017

(30) Foreign Application Priority Data
May 6, 2015 (EP) 15166516.3

(51) Int. Cl. A61K 31/506; A61P 17/02; A61K 31/519; A61K 31/53; A61K 31/5377; A61K 31/197; A61K 31/4439; A61K 31/427; A61K 31/426; A61K 31/635

(52) U.S. Cl. CPC A61K 31/506; A61P 17/02; A61K 31/519; A61K 31/53; A61K 31/635; A61K 31/197; A61K 31/4439; A61K 31/427; A61K 31/426; A61K 31/5377

(57) ABSTRACT

Use of sGC stimulators, sGC activators alone, or in combination with PDE5 inhibitors for the prevention and healing of Digital Ulcers which are concomitant to fibrotic diseases, such as systemic sclerosis and scleroderma.

The use of sGC stimulators, sGC activators alone, or in combination with PDE5 inhibitors for the prevention and healing of Digital Ulcers which are concomitant to fibrotic diseases, such as systemic sclerosis and scleroderma.

### BACKGROUND OF THE INVENTION

### Systemic Sclerosis and Concomitant Digital Ulcers (DU)

The pathogenesis of Systemic Sclerosis (SSc) is still unclear and remains elusive. However, scleroderma is a non-inherited, noninfectious disease and thought to be an autoimmune disease.

SSc has a broad variety of symptoms triggered by excessive deposition of extracellular matrix in the dermis resulting in skin fibrosis. In later stages SSc is characterized by progressive tissue fibrosis affecting other internal organs as the gut, the lung or the kidneys. Therefore scleroderma is the hallmark of the disease comprising also e.g. lung fibrosis, renal fibrosis, fibrosis of the heart, the gut or the blood vessels. Besides excessive fibrosis in the skin and internal organs, SSc is also characterized by vasculopathies and microangiopahties. Especially small vessel vasculopathies and concomitant vascular malperfusion and ischemia can cause Raynaud's phenomena (RP) but also to the formation of digital ulcer (DU). Whereas tissue fibrosis can cause end organ failure and lead to high morbidity and mortality in patients with end-stage SSc, formation of DU substantially reduce the quality of life of SSc patients, impairs hand function and leads to disability. (Harris et al. 2005—Kelley's Textbook of Rhematology 7^{th} edition. Elsevier Saunders, Philadelphia Pa.).

There is still no causative treatment for Systemic Sclerosis (SSc) available and the current therapy is based on suppression of the immune system via corticosteroids, cyclophosphamide, methotrexate. More recently kinase inhibitors and anti-inflammatory drugs are under investigation as immunosuppressant and antifibrotic agents in SSc, but tolerability is limited in SSc patients (Khanna and Denton 2010—Best. Pract. Res. Clin. Rheumatol. 24:387-400, Ong and Denton 2010—Curr. Opin. Rheumatol. 22:264-272, Spiera 2011—Ann. Rheum. Dis. Epub March 2011). These therapies either used as stand alone treatment or combined are of limited efficacy and exhibited considerable side effects. Therefore alternative treatment options in SSc which are efficacious and safe are urgently needed. In addition, there is currently no approved treatment for healing of DU but vasoactive drugs as prostacyclin agonists and endothelin antagonists are used.

Antifibrotic Effects of cGMP:

The cyclic nucleotides, cyclic adenosine monophosphate (cAMP) and cyclic guanosine monophosphate (cGMP), were discovered decades ago and represent one of the most important second messenger pathway within cells. It is well established that the regulation of intra-cellular cGMP pools have substantial impact on physiology, and pathophysiology and is one basic principle of pharmacological intervention (Evgenov et al. 2006—Nat. Rev. Drug. Discov. 5(9):755-768). Besides the treatment of cardiovascular, lung or CNS-disorders there is ample evidence that an increase in cGMP is a very effective treatment option for urological disorders as well (Sandner et al. 2009—Handbook Exper. Pharmacol. 191:507-531). PDE5 inhibitors are the gold-standard for the treatment of erectile dysfunction (ED) but it was shown that PDE5 inhibitors could be useful for the treatment of symptomatic BPH which is characterized by Overactive Bladder (OAB) and Lower Urinary Tract Symptoms (LUTS) (Porst et al. 2008—Curr. Urol. Rep. 9:295-301; McVary et al. 2007—J. Urol. 177:1071-1077, J Urol. 177:1401-1407, Kaplan and Gonzalez. 2007—Rev. Urol. 9:73-77). The antifibrotic effects of Vardenafil, sGC stimulators and sGC activators is not understood yet. There are some descriptions about antifibrotic effects of Nitric-Oxide which are presumably mediated by cGMP in other organs and PDE5 inhibitors or guanylate cyclase stimulators have shown efficacy in penile fibrosis (Peyronie's disease) (Ferrini et al. 2006—B. J. Urol. 97:625-633) and liver fibrosis (Knorr et al. 2008—Arzneimittelforschung 58:71-80) respectively.

It was not known if the NO/cGMP system is involved in SSc and if cGMP increase provides a treatment option for this disease. We hypothetized that—independent from endogenous NO/cGMP production—sGC stimulators and activators might be an effective treatment option for Systemic Sclerosis (SSc) by reduction of skin fibrosis. In WO2011/147810 we have recently shown that sGC stimulators, sGC activators, alone and combinations with PDE5 inhibitors could directly target skin fibrosis which is one hallmark of Systemic Sclerosis (SSc). This clearly demonstrated that sGC stimulators, sGC activators, alone and combinations with PDE5 inhibitors are an effective future treatment option for SSc. However, it is not known if the vasculopathies in SSc patients which lead e.g. to DU formation which are one of the most bothersome symptoms in SSc, could be also efficiently treated with sGC stimulators, sGC activators, alone and combinations with PDE5 inhibitors. Since these compounds can induce peripheral vasodilation it could be assumed that SSc driven vasculopathies might be reduced, preventing new formation of DU. However, it was unclear if SSc-driven DU could be also healed giving the antifibrotic mode of action of sGC stimulators/sGC activators alone and in combination with PDE5 inhibitors. Therefore, increased blood flow may be counteracted by reduced collagen-synthesis or synthesis of extracellular matrix which is necessary for wound closure and which then may impair wound healing in SSc patients. We therefore investigated sGC stimulators and sGC activators, i.e. compound of the formula

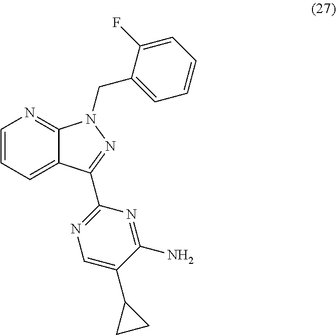 [SMILES: Nc1nc(-c2nn(Cc3ccccc3F)c3ncccc23)ncc1C1CC1]

and combinations with PDE5 inhibitors thereof on wound healing was in TSK mice an animal model for SSc characterized by excessive skin fibrosis We found in vivo in our animal models that:
TSK mice have an attenuated wound healing compared to WT mice.
sGC stimulators or sGC activators, i.e. compounds according to formulae (27) and (3) significantly and dose-dependently accelerated wound healing in the TSK mice.
sGC stimulators or sGC activators, i.e compounds according to formulae (27) and (3) normalized the healing time to healthy WT control mice. These data suggest that despite the antifibrotic effectof sGC stimulators and sGC activators in SSc, wound healing in SSc could be significantly accelerated and normalized to the levels of healthy control individuals

In summary, we found completely unexpected and for the first time that sGC stimulators or sGC activators i.e. compounds according to formulae (27) and (3), which prevent fibrosis and regress established fibrosis in different animal models of inflammatory and non-inflammatory SSc, could also lead to significantly enhanced wound healing in the TSK-mouse SSc model.

Taken together this data indicate for the first time that sGC stimulators and sGC activators, i.e. compounds according to formulae (27) and (3) could improve wound healing in an SSc. These data also suggest that despite the antifibrotic mode of action, these compounds are able to heal DUs in SSc patients.

### DISCLOSURE OF THE INVENTION

Fibrotic disorders addressed by therapeutic agents of the invention which in particular and with substantial advantage can be treated by the above mentioned sGC stimulators or sGC activators alone or in combination with PDE5 inhibitors comprise but are not limited to Systemic Sclerosis (SSc), Systemic Sclerosis (SSc) concomitant fibrosis and fibrotic diseases.

Fibrotic disorders addressed by therapeutic agents of the invention which in particular and with substantial advantage can be treated by the above mentioned sGC stimulators or sGC activators alone or in combination with PDE5 inhibitors comprise but are not limited to Systemic Sclerosis (SSc) concomitant vasculopathies, to Raynaud's phenomena (RD) and the formation and healing of digital ulcers (DU)

Systemic Sclerosis (SSc) refers to but is not limited to diffuse Systemic Sclerosis (dSSc), limited Systemic Sclerosis (1SSc), overlap type of Systemic Sclerosis, undifferentiated type of Systemic Sclerosis, Systemic Sclerosis sine scleroderma, skin fibrosis, scleroderma, nephrogenic fibrosing dermopathy (NFD), nephrogenic systemic fibrosis (NSF), keloid formation.

SSc concomitant fibrosis refers to fibrosis of internal organs, comprising but not limited to the gut, the lung, the kidney and the blood vessels.

Fibrotic diseases comprises but are not limited to a condition in which collagen excess —independent of the etiology i.e. autoimmune disorders, chronic graft versus host disease, radiation therapy, intoxications, diabetes, surgery—lead to fibrosis of the skin, gut, liver, lung, heart, bladder, prostate, blood vessels or any other localized or generalized fibrotic condition in tissues.

In the sense of the present invention, the term fibrotic diseases comprises in particular the following terms: hepatic fibrosis, hepatic cirrhosis, pulmonary fibrosis, endomyocardial fibrosis, nephropathy, glomerulonephritis, interstitial renal fibrosis, fibrotic lesions as a consequence of diabetes, bone marrow fibrosis and similar fibrotic diseases, scleroderma, morphea, keloids, hypertrophic scars (including after surgery), naevi, diabetic retinopathy, proliferative vitreoretinopathy and connective tissue diseases (e.g. sarcoidosis SSc concomitant vasculopathies comprise but are not limited to vascular occlusive diseases vasculitis, micro and macroangiopathies, Raynaud's Phenomena, digital ischemic lesions, digital ulcer, digital necrotic lesions, gangrene anddigital loss.

In the sense of the present invention, sGC stimulators are nitric oxide (NO) independent and heam-dependent modulators of the soluble guanylate cyclase.

In the sense of the present invention, sGC activators are nitric oxide (NO) and heam-independent modulators of the soluble guanylate cyclase.

A preferred embodiment of the invention is the use of compounds according to compounds disclosed in WO03/097063, WO03/09545, WO04/009589, WO03/004503, WO02/070462, WO2007/045366, WO2007/045369, WO2007/045370, WO2007/045433, WO2007/045367, WO2007/124854, WO2007/128454, WO2008/031513, WO2008/061657, WO2008/119457, WO2008/119458, WO2009/127338, WO2010/079120, WO2010/102717, WO2011/051165, WO2011/147809, WO2011/141409, WO2014/012935, WO2012/059549, WO2012/004259, WO2012/004258, WO2012/059548, WO2012/028647, WO2012/152630, WO 2012/076466, WO2014/068099, WO2014/068104, WO2012/143510, WO2012/139888, WO2012/152629, WO2013/004785, WO2013/104598, WO2013/104597, WO2013/030288, WO2013/104703, WO2013/131923, WO2013/174736, WO2014/012934, WO2014/068095, WO2014/195333, WO2014/128109, WO2014/131760, WO2014/131741, WO2015/018808, WO2015/004105, WO2015/018814, WO98/16223, WO98/16507, WO98/23619, WO00/06569, WO01/19776, WO01/19780, WO01/19778, WO02/042299, WO02/092596, WO02/042300, WO02/042301, WO02/036120, WO02/042302, WO02/070459, WO02/070460, WO02/070461, WO02/070510, WO2012/165399, WO2014/084312, WO2011115804, WO2012003405, WO2012064559, WO2014/047111, WO2014/047325, WO2011/149921, WO2010/065275, WO2011/119518 for the manufacture of a medicament for prevention and healing of Digital Ulcers which are concomitant to fibrotic diseases, such as systemic sclerosis and scleroderma. A preferred embodiment of the invention is the use of compounds according to formulae (1)-(28) for the manufacture of a medicament for prevention and healing of Digital Ulcers which are concomitant to fibrotic diseases, such as systemic sclerosis and scleroderma, as shown below:
2-[1-(2-Fluorbenzyl)-1H-pyrazolo[3,4-b]pyridine-3-yl]-5-(4-morpholinyl)-4,6-pyrimidinediamine (1), disclosed as example 16 in WO 00/06569,
2-[1-(2-Fluorobenzyl)-1H-pyrazolo[3,4-b]pyridine-3-yl]-5-(4-pyridinyl)-4-pyrimidine amine (2), disclosed as example 1 in WO 02/42301,
Methyl-4,6-diamino-2-[1-(2-fluorobenzyl)-1H-pyrazolo[3,4-b]pyridine-3-yl]-5-pyrimidinyl-(methyl)carbamate (3), disclosed as example 8 in WO 03/095451,
Methyl-4,6-diamino-2-[1-(2-fluorobenzyl)-1H-pyrazolo[3,4-b]pyridine-3-yl]-5-pyrimidinylcarbamate (4), disclosed as example 5 in WO 03/095451
4-({(4-carboxybutyl) [2-(2-{[4-(2-phenylethyl)benzyl]oxy}phenyl)ethyl]amino}methyl) carboxylic acid (5), disclosed as example 8a in WO 01/019780,
Methyl-{4,6-diamino-2-[5-fluoro-1-(2-fluorbenzyl)-1H-pyrazolo[3,4-b]pyridine-3-yl]pyrimidine-5-yl}carbamate (6), Methyl-{4,6-diamino-2-[5-fluoro-1-(2-fluorbenzyl)-1H-pyrazolo[3,4-b]pyridine-3-yl]pyrimidine-5-yl}methylcarbamate (7), Methyl-{4,6-diamino-2-[5-fluoro-1-(2-fluorbenzyl)-1H-pyrazolo[3,4-b]pyridine-3-yl]pyrimidine-5-yl}(2,2,2-trifluorethyl)carbamate (8), disclosed in WO 2011/147809,
5-Chloro-2-(5-chlorothiophene-2-sulfonylamino-N-(4-(morpholine-4-sulfonyl)-phenyl)-benzamid as sodium salt (9), disclosed in WO00/02851,
2-(4-Chloro-phenylsulfonylamino)-4,5-dimethoxy-N-(4-(thiomorpholine-4-sulfonyl)-phenyl)-benzamide (10), disclosed in WO00/02851,
1-{6-[5-Chloro-2-({4-trans-4-}trifluoromethyl)cyclohexyl]benzyl}oxy)phenyl]pyridine-2-yl}-5-(trifluoromethyl)-1H-pyrazol-4-carboxylic acid (11), disclosed in WO 2009/032249,
1-[6-(2-(2-Methyl-4-(4-trifluoromethoxyphenyl)benzyloxy)-phenyl)pyridine-2-yl]-5-trifluoromethyl-pyrazol-4-carboxylic acid (12), disclosed in WO 2009/071504,
1[6-(3,4-dichlorophenyl)-2-pyridinyl-5-(trifluoromethyl)-1H-pyrazole-4-caboxylic acid (13), disclosed in WO 2009/068652,
1-({2-[3-Chlor-5-(trifluoromethyl)phenyl]-5-methyl-1,3-thiazole-4-yl}methyl)-1H-pyrazole-4-carboxylic acid (14), 4-({2-[3-(Trifluoromethyl)phenyl]-1,3-thiazole-4-yl}methyl)benzoic acid (15) and 1-({2-[2-Fluoro-3-(trifluoromethyl)phenyl]-5-methyl-1,3-thiazole-4-yl}methyl)-1H-pyrazole-4-carboxylic acid (16) disclosed in WO 2009/123316,
4-Amino-2-[5-chloro-3(3,3,3-trifluororpropyl)-1H-indazol-1yl]-5,5-dimethyl-5,7-dihydro-6H-pyrrolo[2,3-d]pyrimidine-6-one (17), 4-Amino-2-[5-chloro-3-(2,3,6-trifluorbenzyle)-1H-indazol-1yl]-5,5-dimethyl-5,7-dihydro-6H-pyrrolo[2,3-d]pyrimidine-6-one (18), 4-Amino-5,5-dimethyl-2-[3-(2,3,6-trifluorbenzyle) 1H-thieno[3,4-c]pyrazol-1-yl]-5,7-dihydro-6H-pyrrolo[2,3-d]pyrimidine-6-one (19), 4-Amino-5,5-dimethyl-2-[3-(2,3,6-trifluorbenzyle)-1H-thieno[2,3-d]pyrazole-1-yl]-5,5-dimethyl-5,7-dihydro-6H-pyrrolo[2,3-d]pyrimidine-6-one (20), 4-Amino-5,5-dimethyl-2-[7-(2,3,6-trifluorobenzyle)imidazo[1,5-b]pyridazine-5-yl]-5,7-dihydro-6H-pyrrolo[2,3-d]pyrimidine-6-one (21), 4-Amino-2-[6-chloro-3-(2,3,6-trifluorobenzyle)imidazo[1,5-a]pyridine-1-yl]]-5,5-dimethyl-5,7-dihydro-6H-pyrrolo[2,3-d]pyrimidine-6-one (22), 4-Amino-2-[6-fluoro-3-(2,3,6-trifluorobenzyle)imidazo[1,5-a]pyridine-1-yl]]-5,5-dimethyl-5,7-dihydro-6H-pyrrolo[2,3-d]pyrimidine-6-one (23), 4-Amino-2-[6-fluoro-3-(2,3,6-trifluorobenzyl)6-fluoroimidazo[1,5-a]pyridine-1-yl]-5,5-dimethyl-5,7-dihydro-6H-pyrrolo[2,3-d]pyrimidine-6-one (24), 4-Amino-5,5-dimethyl-2-[3-(2,4,6-trifluorobenzyle)imidazo[1,5-a]pyridine-1-yl]]-5,7-dihydro-6H-pyrrolo[2,3-d]pyrimidine-6-one (25), 4-Amino-2-[3-(2-cyclopentylethyl)imidazo[1,5-a]pyridine-1-yl]-5,5-dimethyl-5,7-dihydro-6H-pyrrolo[2,3-d]pyrimidine-6-one (26), disclosed in WO 2010/065275,
3-(4-Amino-5-cyclopropylpyrimidine-2-yl)-1-(2-fluorobenzyl)-1H-pyrazolo[3,4-b]pyridine (27) known as BAY 41-2272 disclosed as example 1 in WO 00/06568,
2-{5-Fluor-1-[(3-fluorpyridine-2-yl)methyl]-1H-pyrazolo[3,4-b]pyridine-3-yl}-5-methyl-5-(trifluormethyl)-4-[(3,3,3-trifluorpropyl)amino]-5,7-dihydro-6H-pyrrolo[2,3-d]pyrimidine-6-on (28), disclosed as example 1 in WO 2014/131760.

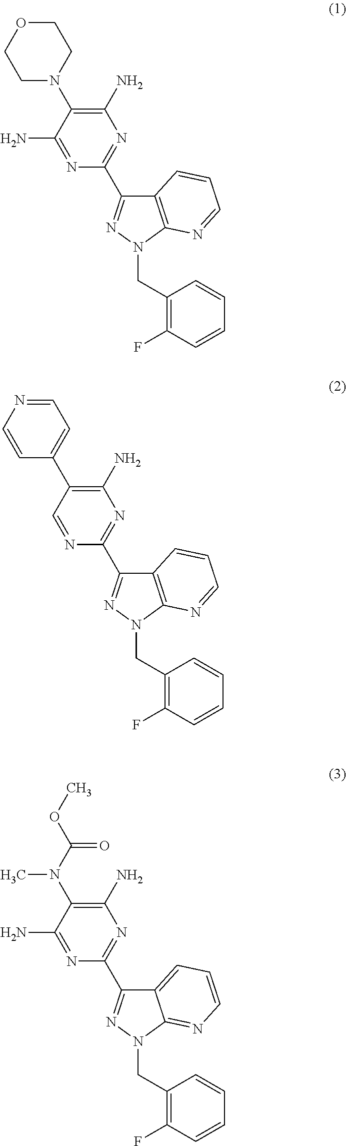 [SMILES: Nc1nc(-c2nn(Cc3ccccc3F)c3ncccc23)nc(N)c1N1CCOCC1 ; Nc1nc(-c2nn(Cc3ccccc3F)c3ncccc23)ncc1-c1ccncc1 ; COC(=O)N(C)c1c(N)nc(-c2nn(Cc3ccccc3F)c3ncccc23)nc1N]

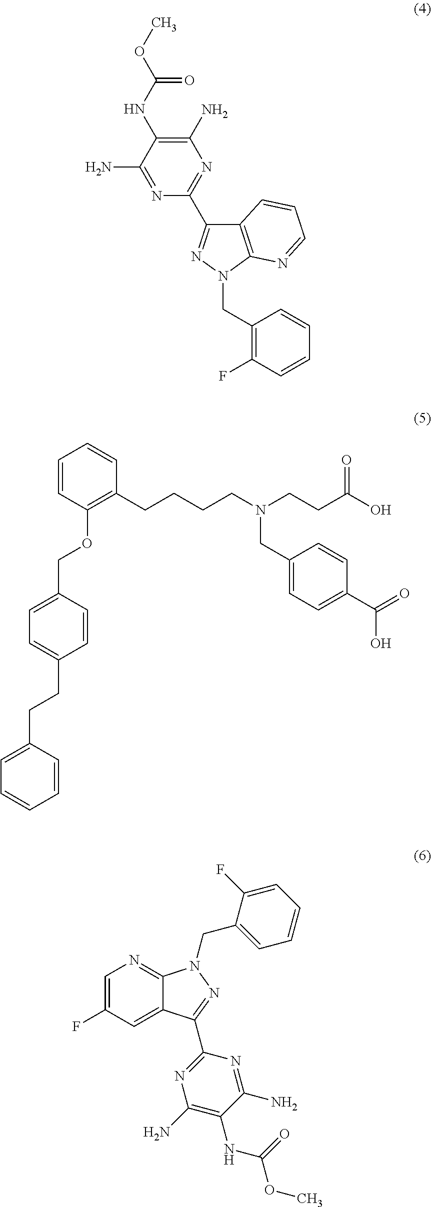 [SMILES: COC(=O)Nc1c(N)nc(-c2nn(Cc3ccccc3F)c3ncccc23)nc1N ; O=C(O)CCN(CCCCc1ccccc1OCc1ccc(CCc2ccccc2)cc1)Cc1ccc(C(=O)O)cc1 ; COC(=O)Nc1c(N)nc(-c2nn(Cc3ccccc3F)c3ncc(F)cc23)nc1N]

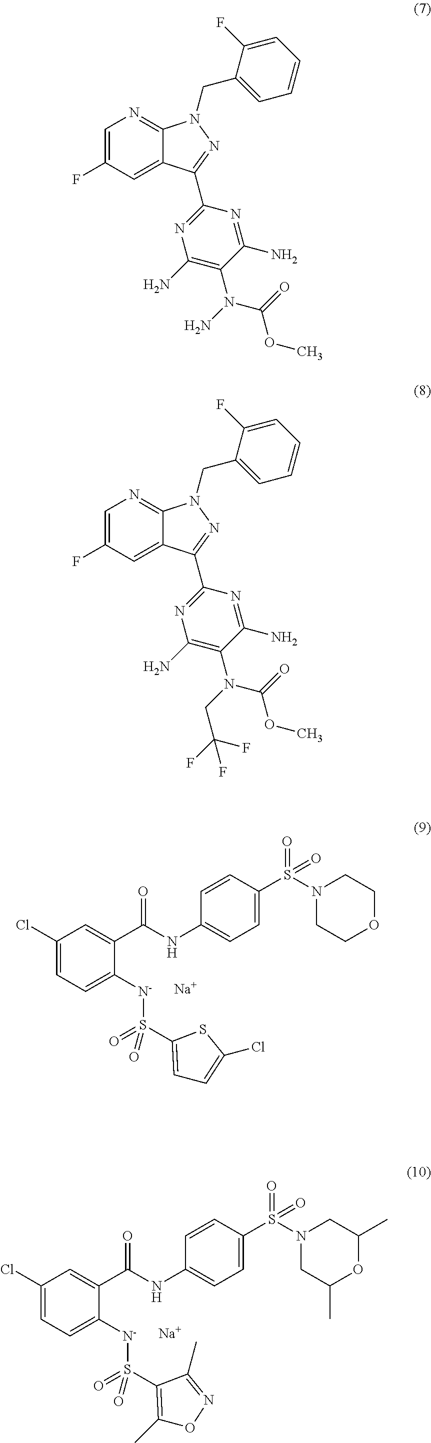 [SMILES: COC(=O)N(N)c1c(N)nc(-c2nn(Cc3ccccc3F)c3ncc(F)cc23)nc1N ; COC(=O)N(CC(F)(F)F)c1c(N)nc(-c2nn(Cc3ccccc3F)c3ncc(F)cc23)nc1N ; O=C(Nc1ccc(S(=O)(=O)N2CCOCC2)cc1)c1cc(Cl)ccc1[N-]S(=O)(=O)c1ccc(Cl)s1 ; [Na+] ; Cc1noc(C)c1S(=O)(=O)[N-]c1ccc(Cl)cc1C(=O)Nc1ccc(S(=O)(=O)N2CC(C)OC(C)C2)cc1 ; [Na+]]

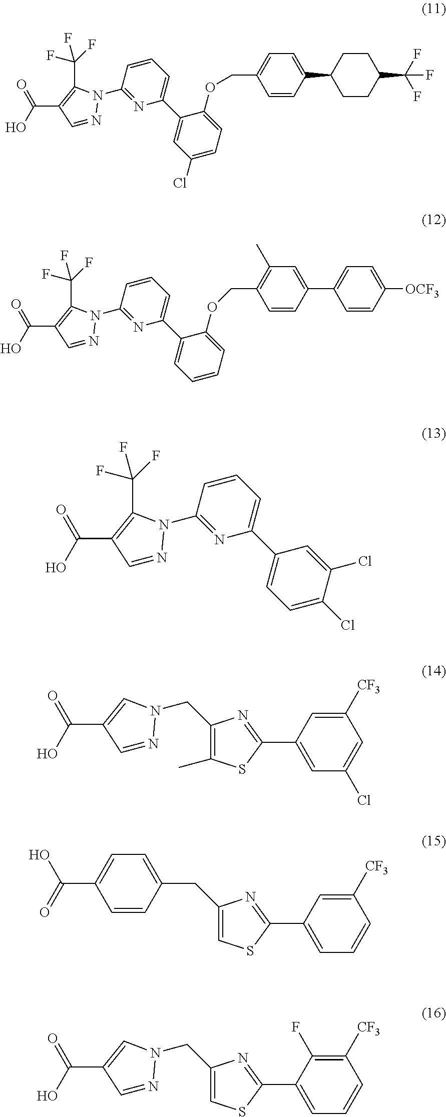

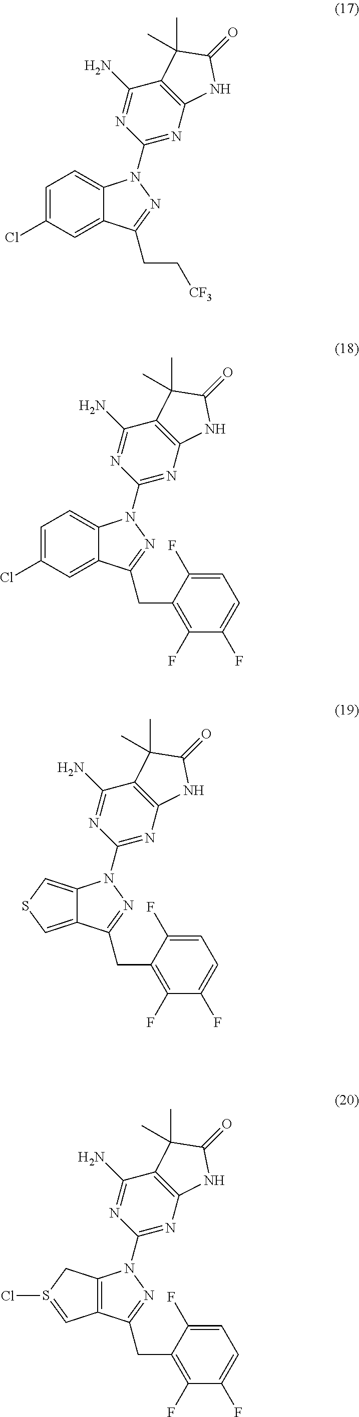 [SMILES: CC1(C)C(=O)Nc2nc(-n3nc(CCC(F)(F)F)c4cc(Cl)ccc43)nc(N)c21 ; CC1(C)C(=O)Nc2nc(-n3nc(Cc4c(F)ccc(F)c4F)c4cc(Cl)ccc43)nc(N)c21 ; CC1(C)C(=O)Nc2nc(-n3nc(Cc4c(F)ccc(F)c4F)c4cscc43)nc(N)c21 ; CC1(C)C(=O)Nc2nc(-n3nc(Cc4c(F)ccc(F)c4F)c4c3CS(Cl)=C4)nc(N)c21]

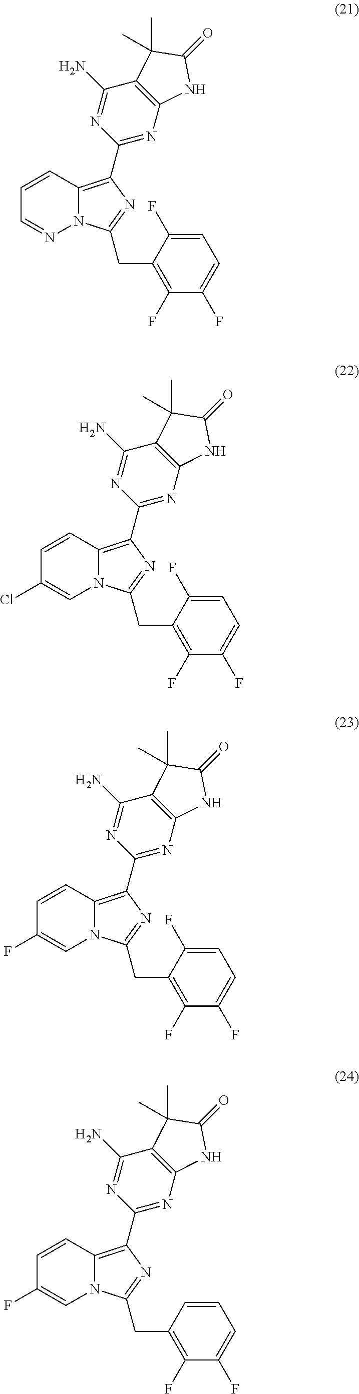 [SMILES: CC1(C)C(=O)Nc2nc(-c3nc(Cc4c(F)ccc(F)c4F)n4ncccc34)nc(N)c21 ; CC1(C)C(=O)Nc2nc(-c3nc(Cc4c(F)ccc(F)c4F)n4cc(Cl)ccc34)nc(N)c21 ; CC1(C)C(=O)Nc2nc(-c3nc(Cc4c(F)ccc(F)c4F)n4cc(F)ccc34)nc(N)c21 ; CC1(C)C(=O)Nc2nc(-c3nc(Cc4cccc(F)c4F)n4cc(F)ccc34)nc(N)c21]

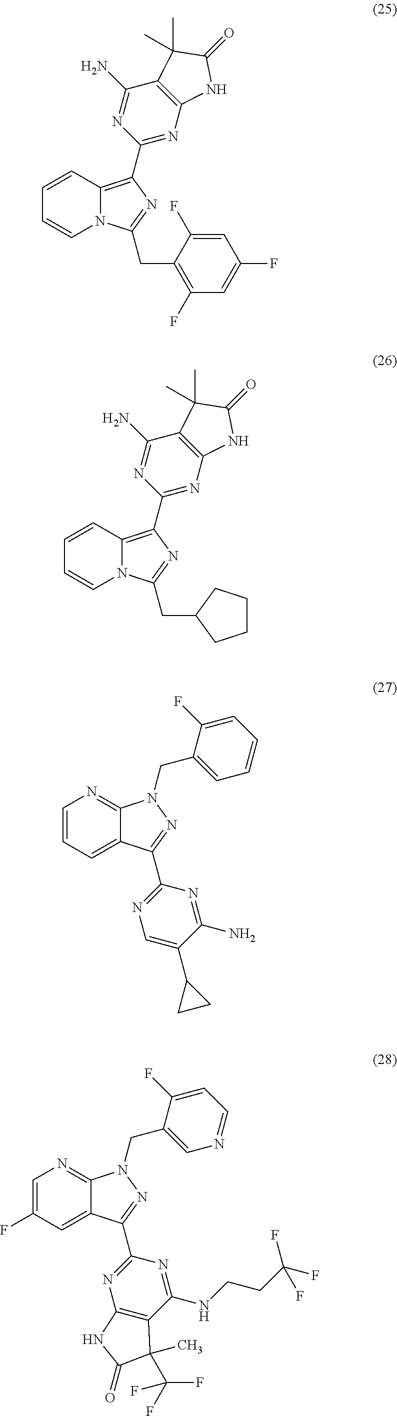 [SMILES: CC1(C)C(=O)Nc2nc(-c3nc(Cc4c(F)cc(F)cc4F)n4ccccc34)nc(N)c21 ; CC1(C)C(=O)Nc2nc(-c3nc(CC4CCCC4)n4ccccc34)nc(N)c21 ; Nc1nc(-c2nn(Cc3ccccc3F)c3ncccc23)ncc1C1CC1 ; CC1(C(F)(F)F)C(=O)Nc2nc(-c3nn(Cc4cnccc4F)c4ncc(F)cc34)nc(NCCC(F)(F)F)c21]

Compounds according to formulae (1), (2), (3), (4), (6)-(8) and (17)-(27) are known as sGC stimulators. Preferred is the use of compounds according to formulae (1), (2), (3), (4), (6), (7), (27) and (28).

Especially preferred is the use of compounds according to formulae (3), (4), (6), (7) and (28).

Especially preferred is the use of compounds according to formulae (3), (4), (6) and (28).

Especially preferred is the use of compounds according to formulae (3), (7) and (28).

Especially preferred is the use of the compound according to formula (3).

Compounds according to formulae (5) und (9)-(16) are known as sGC activators. Preferred is the use of the compound according to formula (5).

A further embodiment of the invention is the use of the combination of stimulators and/or activators of the soluble guanylate cyclase with PDE5 inhibitors for the manufacture of a medicament for prevention and healing of Digital Ulcers which are concomitant to fibrotic diseases, such as systemic sclerosis and scleroderma.

The use of the following PDE 5 inhibitors is preferred for the combination with sGC stimulators and/or activators:

Tadalafil ((6R,12aR)-2,3,6,7,12,12a-Hexahydro-2-methyl-6-(3,4-methylene-dioxyphenyl) pyrazino(1′,2′:1,6)pyrido(3,4-b)indole-1,4-dione), Vardenafil (2-(2-Ethoxy-5-(4-ethylpiperazin-1-yl-1-sulfonyl)phenyl)-5-methyl-7-propyl-3H-imidazo (5,1-f) (1,2,4)triazin-4-one), Sildenafil (3-[2-ethoxy-5-(4-methylpiperazin-1-yl)sulfonyl-phenyl]-7-methyl-9-propyl-2,4,7,8-tetrazabicyclo[4.3.0]nona-3,8,10-trien-5-one), Udenafil 5-[2-propyloxy-5-(1-methyl-2-pyrrolidinylethylamidosulfonyl)phenyl]-methyl-3-propyl-1,6-dihydro-7H-pyrazolo(4,3-d)pyrimidine-7-one, Dasantafil 7-(3-Bromo-4-methoxybenzyl)-1-ethyl-8-[[(1,2)-2-hydroxycyclopentyl]amino]-3-(2-hydroxyethyl)-3,7-dihydro-1-purine-2,6-dione, Avanafil 4-{[(3-chloro-4-methoxyphenyl)methyl]amino}-2-[(2S)-2-(hydroxymethyl)pyrrolidin-1-yl]-N-(pyrimidin-2-ylmethyl)pyrimidine-5-carboxamide, Mirodenafil, Lodenafil, UK 369.003, UK 371.800, SLx 2101 of Surface Logix, LAS 34179 Triazolo[1,2-]xanthine,6-methyl-4-propyl-2-[2-propoxy-5-(4-methylpiperazino)sulfonyl]phenyl or salts, hydrates or hydrates of the salts.

Especially preferred is the use of combinations of compounds according to formulae (1), (2), (3), (4), (6), (7), (27), (28) and/or (5) with vardenafil and/or sildenafil for the manufacture of a medicament in the prevention and healing of Digital Ulcers which are concomitant to fibrotic diseases, such as systemic sclerosis and scleroderma.

Especially preferred is the use of combinations of compounds according to formulae (3), (4), (6), (7), (27), (28) and/or (5) with vardenafil and/or sildenafil for the manufacture of a medicament for the prevention and healing of Digital Ulcers which are concomitant to fibrotic diseases, such as systemic sclerosis and scleroderma.

Especially preferred is the use of compounds according to formulae (3), (4), (6), (7) and/or (28) for the manufacture of a medicament for the prevention and healing of Digital Ulcers which are concomitant to fibrotic diseases, such as systemic sclerosis and scleroderma.

Especially preferred is the use of compounds according to formulae (3), (4) and/or (6) for the manufacture of a medicament for the prevention and healing of Digital Ulcers which are concomitant to fibrotic diseases, such as systemic sclerosis and scleroderma.

Especially preferred is the use of at least one compound according to formulae (3), (4), (6), and/or (7) in combination with vardenafil or sildenafil for the manufacture of a medicament for the prevention and healing of Digital Ulcers which are concomitant to fibrotic diseases, such as systemic sclerosis and scleroderma.

The sGC stimulator such as compound according to formula (3) dose-dependently and significantly accelerated wound healing in the tsk-1 skin fibrosis model in mice. The tsk-1 mouse model is characterized by substantial skin fibrosis reflecting a non-inflammatory driven, stable SSc phenotype. These data imply that the sGC stimulators such as compound according to formula (3) could become an efficacious treatment option for SSc-related vasculopathies, especially for prevention and healing of Digital Ulcer.

The compounds according to the invention can be used alone or in combination with other active substances if necessary. The present invention further relates to medicinal products containing at least one of the compounds according to the invention and one or more further active substances, in particular for the treatment and/or prophylaxis of the aforementioned diseases. As suitable combination active substances, we may mention for example and preferably:
organic nitrates and NO-donors, for example sodium nitroprusside, nitroglycerin, isosorbide mononitrate, isosorbide dinitrate, molsidomine or SIN-1, and inhalational NO;
other vasoactive drugs, for examples prostanoids, such as iloprost, beraprost, cicaprost, epoprostenol, treprostinil;
other vasoactive drugs, for example Rho-kinase inhibitors such as fasudil;
other vasoactive drugs, for example endothelin receptor antagonists such as bosentan, darusentan, ambrisentan or sitaxsentan, macitentan;
active substances for lowering blood pressure, for example and preferably from the group of calcium antagonists, such as nifedipine, amlodipine, verapamil or diltiazem;
active substances for lowering blood pressure, for example and preferably from the group of angiotensin AII antagonists, ACE inhibitors, renin inhibitors, alpha-blockers, beta-blockers, mineralocorticoid receptor antagonists and diuretics; and/or
antithrombotic agents, for example and preferably from the group of platelet aggregation inhibitors, anticoagulants, thrombin inhibitors or profibrinolytic substances;
active substances that alter fat metabolism, for example and preferably from the group of thyroid receptor agonists, cholesterol synthesis inhibitors such as for example and preferably HMG-CoA-reductase or squalene synthesis inhibitors, ACAT inhibitors, CETP inhibitors, MTP inhibitors, PPAR-alpha, PPAR-gamma and/or PPAR-delta agonists, cholesterol absorption inhibitors, lipase inhibitors, polymeric bile acid adsorbers, bile acid reabsorption inhibitors and lipoprotein(a) antagonists;
active substances that are used in fibrotic disorders, for examples and preferable from the group of proteinkinase inhibitors such as sorafenib, regorafenib, imatinib, dasatinib, nilotinib nintedanib, bortezomib and/or pirfenidone;
active substances that alter inflammatory responses and/or suppress immune responses, for example such as, cyclophosphamide, methotrexate, rapamycin, azathioproin, tocilizumab, infliximab, rituximab, adalimumab, belimumab, abatacept, SAR100842, thalidomide derivates;
active substances working on different pathways, for example pirfenidone, SAR100842, thalidomide derivatives, integrin inhibitors.

Another preferred embodiment of the invention are compounds and/or combinations indicated above for use in the prevention and healing of Digital Ulcers which are concomitant to fibrotic diseases, such as systemic sclerosis and/or scleroderma.

Another preferred embodiment of the invention is the use for the production of a medicament for prevention and healing of Digital Ulcers which are concomitant to fibrotic diseases, such as systemic sclerosis and/or scleroderma comprising an effective amount of a compound and/or a combination as indicated above.

Another preferred embodiment of the invention is the pharmaceutical formulation comprising at least one compound or one combination as indicated above for the use in the prevention and healing of Digital Ulcers which are concomitant to fibrotic diseases, such as systemic sclerosis and/or scleroderma.

Another preferred embodiment of the invention is a kit comprising at least one sGC stimulator and/or activator as indicated above or a combination as indicated above for the use in the prevention and healing of Digital Ulcers which are concomitant to fibrotic diseases, such as systemic sclerosis and/or scleroderma.

A pharmaceutical composition of the invention is formulated to be compatible with its intended route of administration. Examples of routes of administration include parenteral e.g., intravenous, intradermal, subcutaneous' oral (e.g., inhalation), transdermal (topical) transmucosal and rectal administration. Pharmaceutical compositions suitable for injectable use include sterile aqueous solutions (where water soluble) or dispersions and sterile powders for the extemporaneous preparation of sterile injectable solutions or dispersions. The carrier can be a solvent or dispersion medium containing, for example, water, ethanol, a pharmaceutically acceptable polyol like glycerol, propylene glycol, liquid polyethylene glycol, and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents for example, parabens, chlorobutanol, phenol, ascorbic acid, thimerosal, and the like. In many cases, it will be preferable to include isotonic agents, for example, sugars, polyalcohols such as maitol sorbitol sodium chloride in the composition.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or con1 starch; a lubricant such as magnesium stearate or sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from a pressurized container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Bio degradable, biocompatible polymers can be used, such as ethylene vinyl acetate, polyanhydrides, polyglycolic acid, collagen, polyorthoesters, and polylactic acid.

### REFERENCES

Beyer C, Schett G, Distler O, Distler J H (2010): Animal models of systemic sclerosis: prospects and limitations. Arthritis Rheum. 62(10):2831-44
Evgenov O V, Pacher P, Schmidt P M et al. (2006): NO-independent stimulators and activators of soluble guanylate cyclase: discovery and therapeutic potential. Nat. Rev. Drug. Discov. 5(9):755-68
Ferrini M G, Kovanecz I, Nolazco G (2006): Effects of long-term vardenafil treatment on the development of fibrotic plaques in a rat model of Peyronie's disease. B. J. U. 97:625-633.
Harris E D, et al. (2005): Kelley's Textbook of Rhematology 7^{th} edition. Elsevier Saunders, Philadelphia Pa.
Kaplan S A, Gonzalez R R (2007): Phosphodiesterase type 5 inhibitors for the treatment of male lower urinary tract symptoms. Rev. Urol. 9(2):73-77
Khanna D and Denton C P (2010) Evidence-based management of rapidly progressing systemic sclerosis. Best. Pract. Res. Clin. Rheumatol. 24:387-400
Knorr A, Hirth-Dietrich C, Alonso-Alija C. et al. (2008): Nitric oxide-independent activation of soluble guanylate cyclase by BAY 60-2770 in experimental liver fibrosis. Arzneimittelforschung 58:71-80.
MVary K K. T. McVary, W. Monnig, J. L. Camps, Jr., J. M. Young, L. J. Tseng and G. van den Ende (2007): Sildenafil citrate improves erectile function and urinary symptoms in men with erectile dysfunction and lower urinary tract symptoms associated with benign prostatic hyperplasia: a randomized, double-blind trial. J. Urol. 177: 1071-1077.
McVary K T, Roehrborn C G, Kaminetsky J C, Auerbach S M, Wachs B, Young J M, Esler A, Sides G D, Denes B S. (2007): Tadalafil relieves lower urinary tract symptoms secondary to benign prostatic hyperplasia. J Urol. 177:1401-1407.
Ong V H and Denton C P (2010): Innovative therapies for systemic sclerosis Curr. Opin. Rheumatol. 22:264-272.
Porst H, Sandner P, Ulbrich E. (2008): Vardenafil in the treatment of lower urinary tract symptoms secondary to benign prostatic hyperplasia. Curr. Urol. Rep. 9:295-301.
Sandner P, Neuser D, Bischoff E (2009): Erectile dysfunction and lower urinary tract. Handb. Exp. Pharmacol. 191:507-531.
Spiera R, Gordon J, Mersten J, Magro C, Mehta M, Wildmann H, Kloiber S, Kirou K, Lyman S, Crow M (2011): Imatinib mesylate (Gleevec) in the treatment of diffuse cutaneous systemic sclerosis: results of a 1 year, phse IIa, single-arm open-label clinical trial. Ann. Rheum. Dis. Epub Mar. 11, 2011

### FIGURES

FIG. 1: Reduction of wound size in WT mice (left) and tsk-1 mice (right) treated with placebo after three days. Data are mean-SEM, n=46 (WT+Placebo) and n=44 (tsk-1+Placebo), */**/***/****=significant with p<0.05/0.01/0.001/0.0001

FIG. 2: Reduction of wound size in tsk-1 mice treated with either placebo or compound according to formula (27), (BAY 41-2272) or compound according to formula (3), (BAY 63-2521) after three days. Data are mean-SEM, n=44-46 (Placebo groups), n=16 (compound according to formula (27) BAY 41-2272 groups) and n=30-32 (compound according to formula (3) BAY 63-2521 groups), */**/***/****=significant with p<0.05/0.01/0.001/0.0001; ns=non significant; BAY41 corresponds to BAY41-2227.

### EXPERIMENTAL PART

### Example A

### Wound Healing in Tsk-1 Mice Versus WT-Mice

The tight-skin (Tsk-1) mouse model of SSc was used to evaluate the effects of compound according to formula (27) and (3) (BAY 41-2272 and BAY 63-2521) on wound healing in mice with substantial skin fibrosis. Due to an autosomal dominant mutation namely a tandem duplication of the fibrillin-1 gene, the phenotype of tsk-1 mice is characterized by an increased hypodermal thickness (Beyer et al. 2010). Genotyping of Tsk-1 mice was performed by PCR with the following primers: mutated fibrillin-1/tsk-1 forward primer: 5′-GTTGGCAACTATACCTGCAT-3′, reverse primer: 5′-CCTTTCCTGGTAACATAGGA-3′.

The effects of placebo (=vehicle for the test compounds=0.5 tylose solution) was studied in either WT mice or in Tsk-1 mice. Tsk-1 mice were anaesthetized and carefully shaved 3 days before setting the wounds for exact quantification of the wound size. In order to avoid influences on wound healing by daily handling of the animals, the usual bi-daily gavage treatment was replaced by drug administration in the food. WT mice and Tsk-1 mice received normal mice chow (placebo) which started on the day of shaving. Three days after shaving, mice were carefully anesthetized and round wounds were punched with 4 mm in diameter. 3 days after punching, mice were euthanized and the wound size was assessed. Statistical analysis of data was done by one-way ANOVA followed by Tuckey's multiple comparison post-hoc analysis

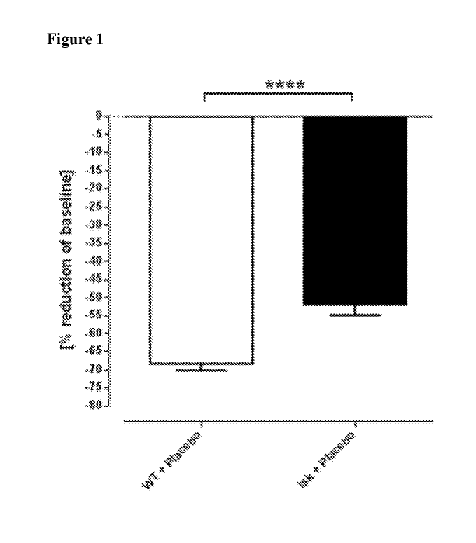

In tsk-1 mice, wound sizes were reduced by 52%±2% after placebo treatment (FIG. 1). In contrast, placebo treated WT mice showed a reduction in wound size of 68%±2% after 3 days (FIG. 1). Therefore, wound healing in tsk-1 mice was partly impaired compared to WT mice and wound closure was significantly attenuated in placebo-treated tsk-1 mice.

Wound Healing in Tsk-1 Mice Treated with the Compound According to Formula (27) and (3), (BAY 41-2272 and BAY 63-2521)

The effects of either placebo (=vehicle for the compounds=0.5% tylose solution), or the compound according to formula (27) or (3), (BAY 41-2272 or BAY 63-2521) were studied in Tsk-1 mice. Tsk-1 mice were anaesthetized and carefully shaved 3 days before setting the wounds for exact quantification of the wound size. In order to avoid influences on wound healing by daily handling of the animals, the bi-daily gavage treatment was replaced by drug administration in the food. Mice received either normal mice chow (placebo) or mice chow, containing 15 and 45 ppm of compound according to formula (27), (BAY 41-2272) or containing 5 ppm, 15 ppm and 45 ppm of compound according to formula (3), (BAY 63-2521), respectively. These dosages—as confirmed by an orientating DMPK-study—resulted in similar exposures as 1 and 3 mg/kg of compound according to formula (27), (BAY 41-2272) BID and 0.3, 1 and 3 mg/kg of compound according to formula (3), (BAY 63-2521) BID, respectively. Treatment groups consist of at least 8 tsk-1 mice per group. Treatment started on the day of shaving to achieve steady state exposure. Three days after shaving, mice were carefully anesthetized and round wounds were punched with 4 mm in diameter. 3 days after punching, mice were euthanized and the wound size was assessed. Statistical analysis of data was done by one-way ANOVA followed by Tuckey's multiple comparison post-hoc analysis

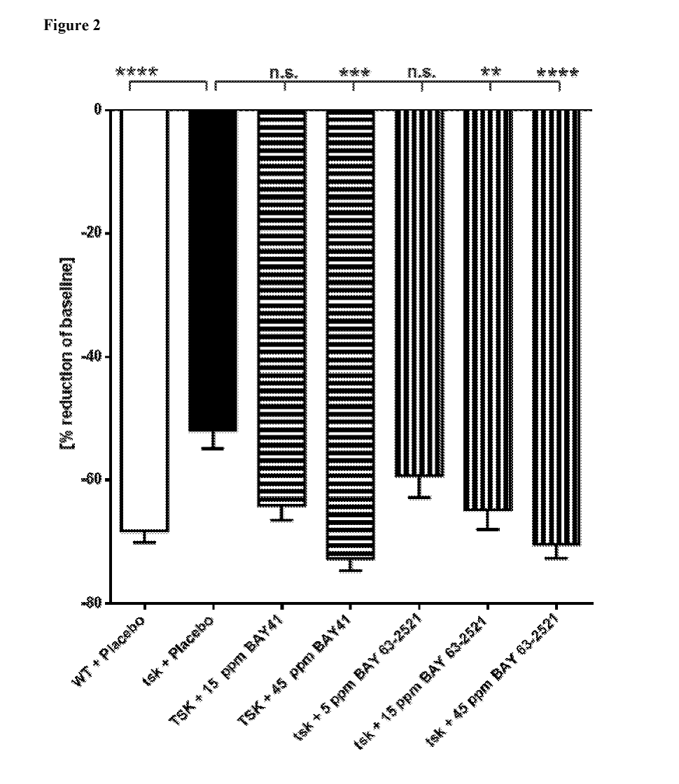

In the tsk-1 mice wound sizes were dose-dependently and significantly reduced by 64±2% and by 73±2% after treatment with 15 and 45 ppm of compound according to formula (27), (BAY 41-2272), respectively (FIG. 2). In addition, in the tsk-1 mice wound sizes were dose-dependently and significantly reduced by 59%±4%, 65±3% and 70%±2% after treatment with 5, 15 and 45 ppm of compound according to formula (3), (BAY 63-2521), respectively (FIG. 2). In addition, treatment with 45 ppm of compound according to formula (27), (BAY 41-2272) and 45 ppm of compound according to formula (3), (BAY 63-2521, Riociguat) normalized wound healing to a similar extent as observed in placebo-treated WT mice (68%±2%) (FIG. 1, FIG. 2). Therefore, compound according to formula (27) and (3), (BAY 41-2272 and BAY 63-2521) accelerated wound healing in compared to placebo treatment in the TSK-mice and lead to a normalization of wound closure as found in healthy control mice.

In summary, these data indicated that:
a) Wound healing in to tsk-1 mice was significantly attenuated compared to WT mice.
b) Wound healing in TSK-1 mice was significantly and dose-dependently improved by treatment with compound according to formula (27), (BAY 41-2272) and/or compound according to formula (3) BAY 63-2521.
Maximum efficacy lead to a wound closure which was similar to healthy WT mice.

Since Tsk-1 mice are an animal model with extensive skin fibrosis reflecting the conditions in SSc this data indicate that the aforementioned compounds could not only reduce fibrosis but also accelerate wound healing in SSc, implying that these compounds are useful for the treatment of DU in SSc patients.

1-12. (canceled)

13. A method for the prevention and healing of digital ulcers that are concomitant to fibrotic diseases comprising administering a therapeutically effective amount of a sGC stimulator or activator alone or in combination with a PDE5 inhibitor to a patient in need thereof.

14. A method for the prevention and healing of digital ulcers that are concomitant to fibrotic diseases, comprising administering a therapeutically effective amount of a sGC stimulator or activator selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), 4-({(4-carboxybutyl)[2-(2-{[4-(2-phenylethyl)benzyl]oxy}phenyl)ethyl]amino}methyl) carboxylic acid (5), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), methyl-{4,6-diamino-2-[5-fluoro-1-(2-fluorobenzyl)-1H-pyrazolo[3,4-b]pyridine-3-yl]pyrimidine-5-yl}(2,2,2-trifluoroethyl)carbamate (8), 5-chloro-2-(5-chlorothiophene-2-sulfonylamino-N-(4-(morpholine-4-sulfonyl)-phenyl)-benzamid as sodium salt (9), 2-(4-chloro-phenylsulfonylamino)-4,5-dimethoxy-N-(4-(thiomorpholine-4-sulfonyl)-phenyl)-benzamide (10), 1-{6-[5-chloro-2-({4-trans-4-}trifluoromethyl)cyclohexyl]benzyl}oxy)phenyl]pyridine-2-yl}-5-(trifluoromethyl)-1H-pyrazol-4-carboxylic acid (11), 1-[6-(2-(2-methyl-4-(4-trifluoromethoxyphenyl)benzyloxy)-phenyl)pyridine-2-yl]-5-trifluoromethyl-pyrazol-4-carboxylic acid (12), 1[6-(3,4-dichlorophenyl)-2-pyridinyl-5-(trifluoromethyl)-1H-pyrazole-4-caboxylic acid (13), 1-({2-[3-chlor-5-(trifluoromethyl)phenyl]-5-methyl-1,3-thiazole-4-yl}methyl)-1H-pyrazole-4-carboxylic acid (14), 4-({2-[3-(trifluoromethyl)phenyl]-1,3-thiazole-4-yl}methyl)benzoic acid (15), 1-({2-[2-fluoro-3-(trifluoromethyl)phenyl]-5-methyl-1,3-thiazole-4-yl}methyl)-1H-pyrazole-4-carboxylic acid (16), 4-amino-2-[5-chloro-3(3,3,3-trifluoropropyl)-1H-indazol-1 yl]-5,5-dimethyl-5,7-dihydro-6H-pyrrolo[2,3-d]pyrimidine-6-one (17), 4-amino-2-[5-chloro-3-(2,3,6-trifluorobenzyl)-1H-indazol-1 yl]-5,5-dimethyl-5,7-dihydro-6H-pyrrolo[2,3-d]pyrimidine-6-one (18), 4-amino-5,5-dimethyl-2-[3-(2,3,6-trifluorobenzyl)1H-thieno[3,4-c]pyrazol-1-yl]-5,7-dihydro-6H-pyrrolo[2,3-d]pyrimidine-6-one (19), 4-amino-5,5-dimethyl-2-[3-(2,3,6-trifluorobenzyl)-1H-thieno[2,3-d]pyrazole-1-yl]-5,5-dimethyl-5,7-dihydro-6H-pyrrolo[2,3-d]pyrimidine-6-one (20), 4-amino-5,5-dimethyl-2-[7-(2,3,6-trifluorobenzyl)imidazo[1,5-b]pyridazine-5-yl]-5,7-dihydro-6H-pyrrolo[2,3-d]pyrimidine-6-one (21), 4-amino-2-[6-chloro-3-(2,3,6-trifluorobenzyl)imidazo[1,5-a]pyridine-1-yl]]-5,5-dimethyl-5,7-dihydro-6H-pyrrolo[2,3-d]pyrimidine-6-one (22), 4-amino-2-[6-fluoro-3-(2,3,6-trifluorobenzyl) imidazo[1,5-a]pyridine-1-yl]]-5,5-dimethyl-5,7-dihydro-6H-pyrrolo[2,3-d]pyrimidine-6-one (23), 4-amino-2-[6-fluoro-3-(2,3,6-trifluorobenzyl)6-fluoroimidazo[1,5-a]pyridine-1-yl]-5,5-dimethyl-5,7-dihydro-6H-pyrrolo[2,3-d]pyrimidine-6-one (24), 4-amino-5,5-dimethyl-2-[3-(2,4,6-trifluorobenzyl)imidazo[1,5-a]pyridine-1-yl]]-5,7-dihydro-6H-pyrrolo[2,3-d]pyrimidine-6-one (25), 4-amino-2-[3-(2-cyclopentylethyl)imidazo[1,5-a]pyridine-1-yl]-5,5-dimethyl-5,7-dihydro-6H-pyrrolo[2,3-d]pyrimidine-6-one (26), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrol[2,3-d]pyrimidin-6-one (28) to a patient in need thereof.

15. The method of claim 14, wherein the digital ulcers are concomitant to fibrotic diseases that are systemic sclerosis or scleroderma.

16. The method of claim 14, wherein sGC stimulator or activator is selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28).

17. The method of claim 16, wherein the digital ulcers are concomitant to fibrotic diseases that are systemic sclerosis or scleroderma.

18. The method of claim 16, wherein the sGC stimulator or activator is selected from the group consisting of methyl-4,6-diamino-2-[1-(2-fluorobenzyl)-1H-pyrazolo[3,4-b]pyridine-3-yl]-5-pyrimidinyl(methyl)carbamate (3), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28).

19. The method of claim 18, wherein the digital ulcers are concomitant to fibrotic diseases that are systemic sclerosis or scleroderma.

20. The method of claim 16, wherein the sGC stimulator or activator is methyl-4,6-diamino-2-[1-(2-fluorobenzyl)-1H-pyrazolo[3,4-b]pyridine-3-yl]-5-pyrimidinyl-(methyl)carbamate (3).

21. The method of claim 20, wherein the digital ulcers are concomitant to fibrotic diseases that are systemic sclerosis or scleroderma.

22. The method of claim 13, wherein a therapeutically effective amount of a sGC stimulator or activator in combination with a PDE5 inhibitor is administered.

23. The method of claim 22, wherein the sGC stimulator or activator is selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28) and the PDE5 inhibitor is selected form the group consisting of vardenafil, sildenafil, tadalafil, udenafil, dasantafil, avanafil, mirodenafil, lodenafil, UK 369.003, UK 371.800, SLx2101 and LAS34179.

24. The method of claim 23, wherein the digital ulcers are concomitant to fibrotic diseases that are systemic sclerosis or scleroderma.

25. The method of claim 23, wherein the sGC stimulator or activator is methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7) or 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28).

26. The method of claim 23, wherein the PDE5 inhibitor is vardenafil or sildenafil.

27. The method of claim 25, wherein the PDE5 inhibitor is vardenafil or sildenafil.

28. A method for accelerating wound healing of digital ulcers that are concomitant to fibrotic diseases, the method comprising administering a therapeutically effective amount of a sGC stimulator or activator alone or in combination with a PDE5 inhibitor to a patient in need thereof.

29. A method for accelerating wound healing of digital ulcers that are concomitant to fibrotic diseases, the method comprising administering a therapeutically effective amount of a sGC stimulator or activator selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), 4-({(4-carboxybutyl)[2-(2-{[4-(2-phenylethyl)benzyl]oxy}phenyl)ethyl]amino}methyl) carboxylic acid (5), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), methyl-{4,6-diamino-2-[5-fluoro-1-(2-fluorobenzyl)-1H-pyrazolo[3,4-b]pyridine-3-yl]pyrimidine-5-yl}(2,2,2-trifluoroethyl)carbamate (8), 5-chloro-2-(5-chlorothiophene-2-sulfonylamino-N-(4-(morpholine-4-sulfonyl)-phenyl)-benzamid as sodium salt (9), 2-(4-chloro-phenylsulfonylamino)-4,5-dimethoxy-N-(4-(thiomorpholine-4-sulfonyl)-phenyl)-benzamide (10), 1-{6-[5-chloro-2-({4-trans-4-}trifluoromethyl)cyclohexyl]benzyl}oxy)phenyl]pyridine-2-yl}-5-(trifluoromethyl)-1H-pyrazol-4-carboxylic acid (11), 1-[6-(2-(2-methyl-4-(4-trifluoromethoxyphenyl)benzyloxy)-phenyl)pyridine-2-yl]-5-trifluoromethyl-pyrazol-4-carboxylic acid (12), 1[6-(3,4-dichlorophenyl)-2-pyridinyl-5-(trifluoromethyl)-1H-pyrazole-4-carboxylic acid (13), 1-({2-[3-chlor-5-(trifluoromethyl)phenyl]-5-methyl-1,3-thiazole-4-yl}methyl)-1H-pyrazole-4-carboxylic acid (14), 4-({2-[3-(trifluoromethyl)phenyl]-1,3-thiazole-4-yl}methyl)benzoic acid (15), 1-({2-[2-fluoro-3-(trifluoromethyl)phenyl]-5-methyl-1,3-thiazole-4-yl}methyl)-1H-pyrazole-4-carboxylic acid (16), 4-amino-2-[5-chloro-3(3,3,3-trifluoropropyl)-1H-indazol-1 yl]-5,5-dimethyl-5,7-dihydro-6H-pyrrolo[2,3-d]pyrimidine-6-one (17), 4-amino-2-[5-chloro-3-(2,3,6-trifluorobenzyl)-1H-indazol-1yl]-5,5-dimethyl-5,7-dihydro-6H-pyrrolo[2,3-d]pyrimidine-6-one (18), 4-amino-5,5-dimethyl-2-[3-(2,3,6-trifluorobenzyl) 1H-thieno[3,4-c]pyrazol-1-yl]-5,7-dihydro-6H-pyrrolo[2,3-d]pyrimidine-6-one (19), 4-amino-5,5-dimethyl-2-[3-(2,3,6-trifluorobenzyl)-1H-thieno[2,3-d]pyrazole-1-yl]-5,5-dimethyl-5,7-dihydro-6H-pyrrolo[2,3-d]pyrimidine-6-one (20), 4-amino-5,5-dimethyl-2-[7-(2,3,6-trifluorobenzyl)imidazo[1,5-b]pyridazine-5-yl]-5,7-dihydro-6H-pyrrolo[2,3-d]pyrimidine-6-one (21), 4-amino-2-[6-chloro-3-(2,3,6-trifluorobenzyl)imidazo[1,5-a]pyridine-1-yl]]-5,5-dimethyl-5,7-dihydro-6H-pyrrolo[2,3-d]pyrimidine-6-one (22), 4-amino-2-[6-fluoro-3-(2,3,6-trifluorobenzyl)imidazo[1,5-a]pyridine-1-yl]]-5,5-dimethyl-5,7-dihydro-6H-pyrrolo[2,3-d]pyrimidine-6-one (23), 4-amino-2-[6-fluoro-3-(2,3,6-trifluorobenzyl)6-fluoroimidazo[1,5-a]pyridine-1-yl]-5,5-dimethyl-5,7-dihydro-6H-pyrrolo[2,3-d]pyrimidine-6-one (24), 4-amino-5,5-dimethyl-2-[3-(2,4,6-trifluorobenzyl)imidazo[1,5-a]pyridine-1-yl]]-5,7-dihydro-6H-pyrrolo[2,3-d]pyrimidine-6-one (25), 4-amino-2-[3-(2-cyclopentylethyl)imidazo[1,5-a]pyridine-1-yl]-5,5-dimethyl-5,7-dihydro-6H-pyrrolo[2,3-d]pyrimidine-6-one (26), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28) to a patient in need thereof.

30. The method of claim 29, wherein the digital ulcers are concomitant to fibrotic diseases that are systemic sclerosis or scleroderma.

31. The method of claim 29, wherein the sGC stimulator or activator is selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), methyl-{4,6-diamino-2-[5-fluoro-1-(2-fluorobenzyl)-1H-pyrazolo[3,4-b]pyridine-3-yl]pyrimidine-5-yl}carbamate (6), methyl-{4,6-diamino-2-[5-fluoro-1-(2-fluorobenzyl)-1H-pyrazolo[3,4-b]pyridin-3-yl]pyrimidine-5-yl}methylcarbamate (7), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28).

32. The method of claim 28, wherein a therapeutically effective amount of a sGC stimulator or activator in combination with a PDE5 inhibitor is administered.

33. The method of claim 32, wherein the sGC stimulator or activator is selected from the group consisting of 2-[1-(2-fluorobenzyl)-1H-pyrazolo[3,4-b]pyridine-3-yl]-5-(4-morpholinyl)-4,6-pyrimidinediamine (1), 2-[1-(2-fluorobenzyl)-1H-pyrazolo[3,4-b]pyridine-3-yl]-5-(4-pyridinyl)-4-pyrimidine amine (2), methyl-4,6-diamino-2-[1-(2-fluorobenzyl)-1H-pyrazolo[3,4-b]pyridine-3-yl]-5-pyrimidinyl(methyl)carbamate (3), methyl-4,6-diamino-2-[1-(2-fluorobenzyl)-1H-pyrazolo[3,4-b]pyridine-3-yl]-5-pyrimidinylcarbamate (4), methyl-{4,6-diamino-2-[5-fluoro-1-(2-fluorobenzyl)-1H-pyrazolo[3,4-b]pyridin-3-yl]pyrimidine-5-yl}carbamate (6), methyl-{4,6-diamino-2-[5-fluoro-1-(2-fluorobenzyl)-1H-pyrazolo[3,4-b]pyridine-3-yl]pyrimidine-5-yl}methylcarbamate (7), 3-(4-amino-5-cyclopropylpyrimidine-2-yl)-1-(2-fluorobenzyl)-1H-pyrazolo[3,4-b]pyridine (27), and 2-{5-fluoro-1-[(3-fluoropyridin-2-yl)methyl]-1H-pyrazolo[3,4-b]pyridin-3-yl}-5-methyl-5-(trifluoromethyl)-4-[(3,3,3-trifluoropropyl)amino]-5,7-dihydro-6H-pyrrolo[2,3-d]pyrimidin-6-one (28) and the PDE5 inhibitor is selected form the group consisting of vardenafil, sildenafil, tadalafil, udenafil, dasantafil, avanafil, mirodenafil, lodenafil, UK 369.003, UK 371.800, SLx2101 and LAS34179.


SEQUENCE LISTING (2 sequences)

SEQ ID NO: 1 - DNA, 20 bp, Artificial Sequence
  mutated fibrillin-1/ tsk-1 forward primer
gttggcaactatacctgcat

SEQ ID NO: 2 - DNA, 20 bp, Artificial Sequence
  reverse primer
cctttcctggtaacatagga